(19) United States
(12) Patent Application Publication
(10) Pub. No.: US 2010/0166865 A1
(43) Pub. Date: Jul. 1, 2010

(54) NANOPARTICLE COMPOSITIONS

(75) Inventors: Saran Kumar, Edison, NJ (US); Wen-Chung Shieh, Berkeley Heights, NJ (US); Seema Tomer, Parsippany, NJ (US); Joseph Lawrence Zielinski, Florham Park, NJ (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 12/376,458

(22) PCT Filed: Aug. 15, 2007
(86) PCT No.: PCT/US07/75968

§ 371 Date: Feb. 5, 2009

Related U.S. Application Data
(60) Provisional application No. 60/822,674, filed on Aug. 17, 2006.

(51) Int. Cl. A61K 31/675; A61K 9/14; A61P 35/00

(52) U.S. Cl. 424/486; 514/94; 977/773; 977/915; 977/906

(57) ABSTRACT

A method of Nanoparticle-based therapy for a mammalian subject is disclosed. The method uses Nanoparticles and/or Nanoparticles with outer surfaces that contain an affinity moiety effective to bind specifically to a biological surface at which the therapy is aimed, and a hydrophilic polymer coating. The hydrophilic polymer coating is made up of polymer chains either covalently linked or surface adsorbed to the polymer components. After a desired Nanoparticle biodistribution is achieved, the affinity agent binds to the target surface and helps internalize the Nanoparticles

### FIELD OF THE INVENTION

The present invention relates to a therapeutic composition and method that employs, as the delivery vehicle, Nanoparticle formulations. The Nanoparticles optionally comprise of an affinity moiety on the outer Nanoparticle surfaces for effective binding and internalization by target tissues. The Nanoparticles optionally also comprise a surface coating of hydrophilic polymers for steric stability and prolonged circulation.

### BACKGROUND OF THE INVENTION

Nanoparticles can be used for a variety of therapeutic purposes, in particular, for carrying therapeutic agents to target cells by systemic administration of Nanoparticles.

For a variety of reasons, it may be desirable to shield a therapeutic agent using a Nanoparticle. In order to exploit the therapeutic effects of the bisphosphonate class of drugs, the drug distribution must be altered in a way so the therapeutic agent can effectively interact specifically to a target surface at which the therapy is aimed. Therefore, it is desirable to provide a therapeutic Nanoparticle composition.

### SUMMARY OF THE INVENTION

In one aspect, the invention includes a method of Nanoparticle-based therapy for a mammalian subject which includes systemically administering to the subject, Nanoparticles containing:

(i) a polymer matrix; and

(ii) a therapeutic agent.

The polymer matrix provides protection of a therapeutic agent which otherwise will be in a solution form in traditional formulations and will rapidly distribute to the entire body.

Vesicle based Nanoparticles, such as liposomal formulations are another approach to administering therapeutic agents for targeted drug delivery. In case of bisphosphonates, it was surprisingly found that such vesicular formulations can actually cause hypocalcemia due to sequestration of calcium within the vesicle from the surrounding medium after systemic administration. This may eventually lead to toxicity (reference: Liposome patent). Such sequestration of calcium ions in case of polymer matrix based Nanoparticles, as described in this invention, will be avoided and thus such formulations are expected to offer superior safety relative to vesicle based systems.

Another aspect, the invention includes a method of Nanoparticle-based therapy for a mammaliam subject which includes systemically administering to the subject Nanoparticles containing:
(i) a polymer matrix;
(ii) a therapeutic agent;
(iii) a hydrophilic polymer coating for steric stability and prolonged circulation; and, optionally
(iv) an affinity moiety effective to bind specifically to a target surface at which the therapy is aimed.
   The hydrophilic polymer coating is made up of polymer chains which are either covalently linked to surface components of the polymer matrix in the Nanoparticles or adsorbed on the polymer matrix surface by charge interactions.

In one embodiment the polymer matrix contains calcium ions.

In one embodiment, where a therapeutic agent is to be administered to a target region, the affinity moiety is a ligand effective to bind specifically with a receptor at the target region, and the Nanoparticles include the therapeutic agent in entrapped form. An example of this embodiment is treatment of a solid tumor, where the affinity moiety is effective to bind specifically to a tumor-specific receptor or antigen, the Nanoparticles have an average size between about 10 nm to about 500 nm and include an entrapped drug.

In one embodiment the polymer matrix contains copolymers of lactic and glycolic acids.

### DETAILED DESCRIPTION OF THE INVENTION

### I. Nanoparticle Composition

A Nanoparticle for use in Nanoparticle-based therapy, has at least one outer layer having an outer surface. It will be appreciated that the Nanoparticle may include additional layers. In one case, the outer layer is either composed of covalently linked hydrophilic polymer that in turn is covalently linked to a targeting moiety. In another case, the outer layer consisting of a hydrophilic polymer covalently linked to a targeting moiety on one end and in addition covalently linked, as well as by electrostatic interactions to a charge moiety on the other end. The charge moiety is selected from various amino acids or amino acid based polymers that has an opposite charge to that of the polymer matrix.

The Nanoparticle comprises a polymer matrix containing a divalent cation to effectively shield the therapeutic agent from leaching out before it is exposed for interaction with its target. The divalent cation matrix increases the encapsulation efficiency and drug loading of the therapeutic agent and decreases the permeability of the therapeutic agent across the Nanoparticle by trapping the drug. A divalent cation matrix assists in trapping therapeutic agents that are highly soluble. In addition, a divalent cation matrix can facilitate therapeutic agents delivery to tumor more efficiently.

In one embodiment, calcium ions incorporated into the Nanoparticle helps to retain the active drug from dispersing before reacting the target.

A therapeutic agent to be administered to a target cell or region is entrapped in a Nanoparticle. As used herein, therapeutic agent, compound and drug are used interchangeably.

The entrapped therapeutic agent may be any of a large number of therapeutic agents that can be entrapped in polymer matrices, including water-soluble agents, lipophilic compounds, or agents that can be stably attached, e.g., by electrostatic attachment to the outer vesicle surfaces. Exemplary water-soluble compounds include the bisphosphonate class of drugs. Examples of a therapeutic agent are substituted alkanediphosphonic acids, in particular to heteroarylalkanediphosphonic acids of formula (I):

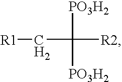

wherein
R1 is a 5-membered heteroaryl radical which contains, as hetero atoms, 2-4 N-atoms or 1 or 2 N-atoms, as well as 1 O- or S-atom, and which is unsubstituted or C-substituted by lower alkyl, phenyl or phenyl which is substituted by lower alkyl, lower alkoxy and/or halogen, or by lower alkoxy, hydroxy, di-lower alkylamino, lower alkylthio and/or halogen, and/or is N-substituted at a N-atom which is capable of substitution by lower alkyl, lower alkoxy and/or halogen; and
R2 is hydrogen, hydroxy, amino, lower alkylthio or halogen, and to the salts thereof, to the preparation of said compounds, to pharmaceutical compositions containing them, and to the use thereof as medicaments.

Examples of 5-membered heteroaryl radicals containing 2-4 N-atoms or 1 or 2 N-atoms, as well as 1 O- or S-atom as hetero atoms are imidazolyl, e.g., imidazol-1-yl, imidazol-2-yl or imidazol-4-yl, pyrazolyl, e.g., pyrazol-1-yl or pyrazol-3-yl, thiazolyl, e.g., thiazol-2-yl or thiazol-4-yl, or, less preferably, oxazolyl, e.g., oxazol-2-yl or oxazol-4-yl, isoxazolyl, e.g., isooxazol-3-yl or isooxazol-4-yl, triazolyl, e.g., 1H-1,2,4-triazol-1-yl, 4H-1,2,4-triazol-3-yl or 4H-1,2,4-triazol-4-yl or 2H-1,2,3-triazol-4-yl, tetrazolyl, e.g., tetrazol-5-yl, thiadiazolyl, e.g., 1,2,5-thiadazol-3-yl, and oxdiazolyl, e.g., 1,3,4-oxadiazol-2-yl. These radicals may contain one or more identical or different, preferably one or two identical or different, substituents selected from the group mentioned at the outset. Radicals R1, unsubstituted or substituted as indicated, are, e.g., imidazol-2-yl or imidazol-4-yl radicals which are unsubstituted or C-substituted by phenyl or phenyl which is substituted as indicated, or which are C- or N-substituted by C₁-C₄alkyl, e.g., methyl, and are typically imidazol-2-yl, 1-C₁-C₄alkylimidazol-2-yl, such as 1-methylimidazol-2-yl, or 2- or 5-C₁-C₄alkylimidazol-4-yl, such as 2- or 5-methylimidazol-4-yl, unsubstituted thiazolyl radicals, e.g., thiazol-2-yl, or 1H-1,2,4-triazol radicals, unsubstituted or substituted by C₁-C₄alkyl, such as methyl, e.g., 1-C₁-C₄alkyl-1H-1,2,4-triazol-5-yl, such as 1-methyl-1H-1,2,4-triazol-5-yl, or imidazol-1-yl, pyrazolyl-1-yl, 1H-1,2,4-triazol-1-yl, 4H-1,2,4-triazol-4-yl or tetrazol-1-yl radicals, unsubstituted or C-substituted by phenyl or phenyl which is substituted as indicated or by C₁-C₄alkyl, such as methyl, e.g., imidazol-1-yl, 2-, 4- or 5-C₁-C₄alkylimidazol-1-yl, such as 2-, 4- or 5-methylimidazol-1-yl, pyrazol-1-yl, 3- or 4-C₁-C₄alkylpyrazol-1-yl, such as 3- or 4-methylpyrazol-1-yl, 1H-1,2,4-tetrazol-1-yl, 3-C₁-C₄alkyl-1H-1,2,4-triazol-1-yl, such as 3-methyl-1H-1,2,4-triazol-1-yl, 4H-1,2,4-triazol-1-yl, 3-C₁-C₄alkyl-4H-1,2,4-traizol-4-yl, such as 3-methyl-4H-1,2,4-triazol-4-yl or 1H-1,2,4-tetrazol-1-yl.

Radicals and compounds hereinafter qualified by the term “lower” will be understood as meaning typically those containing up to 7 carbon atoms inclusive, preferably up to 4 carbon atoms inclusive. The general terms have, e.g., the following meanings:

Lower alkyl is, e.g., C₁-C₄alkyl, such as methyl, ethyl, propyl or butyl, and also isobutyl, sec-butyl or tert-butyl, and may further be C₅-C₇alkyl, such as pentyl, hexyl or heptyl.

Phenyl-lower alkyl is, e.g., phenyl-C₁-C₄alkyl, preferably 1-phenyl-C₁-C₄alkyl, such as benzyl.

Lower alkoxy is, e.g., C₁-C₄alkoxy, such as methoxy, ethoxy, propoxy, isopropoxy, butoxy, isobutoxy, sec-butoxy or tert-butoxy.

Di-lower alkylamino is, e.g., di-C₁-C₄alkylamino, such as dimethylamino, diethylamino. N-ethyl-N-methylamino, dipropylamino, N-methyl-N-propylamino or dibutylamino.

Lower alkylthio is, e.g., C₁-C₄alkylthio, such as methylthio, ethylthio, propylthio or butylthio, and also isobutylthio, sec-butylthio or tert-butylthio.

Halogen is, e.g., halogen having an atomic number of up to 35 inclusive, such as fluorine, chlorine or bromine.

Salts of compounds of formula (I) are in particular the salts thereof with pharmaceutically acceptable bases, such as non-toxic metal salts derived from metals of groups Ia, Ib, IIa and IIb, e.g., alkali metal salts, preferably sodium or potassium salts, alkaline earth metal salts, preferably calcium or magnesium salts, copper, aluminum or zinc salts, and also ammonium salts with ammonia or organic amines or quaternary ammonium bases, such as free or C-hydroxylated aliphatic amines, preferably mono-, di- or tri-lower alkylamines, e.g., methylamine, ethylamine, dimethylamine or diethylamine, mono-, di- or tri(hydroxy-lower alkyl)amines such as ethanolamine, diethanolamine or triethanolamine, tris(hydroxymethyl)aminomethane or 2-hydroxy-tert- butylamine, or N-(hydroxy-lower alkyl)-N,N-di-lower alkylamines or N-(polyhydroxy-lower alkyl)-N-lower alkylamines, such as 2-(dimethylamino)ethanol or D-glucamine, or quaternary aliphatic ammonium hydroxides, e.g., with tetrabutylammonium hydroxide.

In this connection it should also be mentioned that the compounds of formula (I) may also be obtained in the form of inner salts, provided the group R1 is sufficiently basic. These compounds can therefore also be converted into the corresponding acid addition salts by treatment with a strong protic acid such as a hydrohalic acid, sulfuric acid, sulfonic acid, e.g., methanesulfonic acid or p-toluenesulfonic acid, or sulfamic acid, e.g., N-cyclohexylsulfamic acid.

In one embodiment, the therapeutic agents are compounds of formula (I),

wherein
R1 is an imidazolyl, pyrazolyl, 2H-1,2,3-triazolyl, 1H-1,2,4-triazolyl or 4H-1,2,4triazolyl, tetrazolyl, oxazolyl, isoxazolyl, oxadiazolyl, thiazolyl or thiadiazolyl radical which is unsubstituted or C-substituted by one or two members selected from lower alkyl, lower alkoxy, phenyl or phenyl which is in turn substituted by one or two members selected from lower alkyl, lower alkoxy and/or halogen, hydroxy, di-lower alkylamino, lower alkylthio and/or halogen, and/or is N-substituted at a N-atom which is capable of substitution by lower alkyl or phenyl-lower alkyl which is unsubstituted or substituted by one or two members selected from lower alkyl, lower alkoxy and/or halogen; and
R2 is hydrogen, hydroxy, amino, lower alkylthio or halogen, and salts thereof, especially the inner salts and pharmaceutically acceptable salts thereof with bases.

In one embodiment, the therapeutic agents are compounds of formula (I),

wherein
R1 is an imidazolyl, pyrazolyl, 2H-1,2,3-triazolyl or 4H-1,2,4-triazolyl, tetrazolyl, oxazolyl, isoxazolyl, oxadiazolyl, thiazolyl or thiadiazolyl radical which is unsubstituted or C-substituted by one or two members selected from lower alkyl, lower alkoxy, phenyl or phenyl which is in turn substituted by one or two members selected from lower alkyl, lower alkoxy and/or halogen, hydroxy, di-lower alkylamino, lower alkylthio and/or halogen, and/or is N-substituted at a N-atom which is capable of substitution by lower alkyl or phenyl-lower alkyl which is unsubstituted or substituted by one or two members selected from lower alkyl, lower alkoxy and/or halogen; and
R2 is hydrogen, hydroxy, amino, lower alkylthio or halogen, and salts thereof, especially the inner salts and pharmaceutically acceptable salts thereof with bases.

In one embodiment, the therapeutic agents are compounds of formula (I),

wherein
R1 is an imidazolyl radical, such as imidazo -1-yl, imidazol-2-yl or imidazol-4-yl, a 4H-1,2,4-triazolyl radical, such as 4H-1,2,4-triazol-4-yl, or a thiazolyl radical, such as thiazol-2-yl, which radical is unsubstituted or C-substituted by one or two members selected from C₁-C₄alkyl, such as methyl, C₁-C₄alkoxy, such as methoxy, phenyl, hydroxy, di-C₁-C₄alkylamino, such as dimethylamino or diethylamino, C₁-C₄alkylthio, such as methylthio, and/or halogen having an atomic number up to 35 inclusive, such as chlorine, and/or is N-substituted at a N-atom which is capable of substitution by C₁-C₄alkyl, such as methyl, or phenyl-C₁-C₄alkyl, such as benzyl; and
R2 is preferably hydroxy or, less preferably, hydrogen or amino, and salts thereof, especially the inner salts and pharmaceutically acceptable salts thereof with bases.

In one embodiment, the therapeutic agents are compounds of formula (I),

wherein
R1 is an imidazol-2- or -4-yl radical which is unsubstituted or C-substituted by phenyl or C- or N-substituted by C₁-C₄alkyl, such as methyl, e.g., imidazol-2-yl, 1-C₁-C₄alkylimidazol-2-yl, such as 1-methylimidazol-2-yl, or 2- or 5-C₁-C₄alkylimidazol-4-yl, such as 2- or 5-methylimidazol-4-yl, or is an unsubstituted thiazolyl radical, e.g., thiazol-2-yl, or is a 1H-1,2,4-triazolyl radical which is unsubstituted or substituted by C₁-C₄alkyl, such as methyl, e.g., 1-C₁-C₄alkyl-1H-1,2,4-triazol-5-yl, such as 1-methyl-1H-1,2,4-triazol-5-yl; and
R2 is hydroxy or, less preferably, hydrogen, and salts, especially pharmaceutically acceptable salts, thereof.

In one embodiment, the therapeutic agents are compounds of formula (I),

wherein
R1 is an imidazol-1-yl, pyrazol-1-yl, 1H-1,2,4-triazol-1-yl, 4H-1,2,4-triazol-4-yl or tetrazol-1-yl radical which is unsubstituted or C-substituted by phenyl or C₁-C₄alkyl, such as methyl, e.g., imidazol-1-yl, 2-, 4- or 5-C₁-C₄alkylimidazol-1-yl, such as 2-, 4- or 5-methylimidazol-1-yl, pyrazol-1-yl, 3- or 4-C₁-C₄alkylpyrazol-1-yl, such as 3- or 4-methylpyrazol-1-yl, 1H-1,2,4-tetrazol-1-yl, 3-C₁-C₄alkyl-1H-1,2,4-triazol-1-yl, such as 3-methyl-1H-1,2,4-triazol-1-yl, 4H-1,2,4-triazol-1-yl, 3-C₁-C₄alkyl-4H-1,2,4-triazol-4-yl, such as 3-methyl-4H-1,2,4-triazol-4-yl or 1H-tetrazol-1-yl; and
R2 is hydroxy or, less preferably, hydrogen, and salts, especially pharmaceutically acceptable salts, thereof.

In one embodiment, the therapeutic agents are compounds of formula (I),

wherein
R1 is an imidazolyl radical which is unsubstituted or substituted by C₁-C₄alkyl, such as methyl, e.g., imidazol-1-yl, imidazol-2-yl, 1-methylimidazol-2-yl, imidazol-4-yl or 2- or 5-methylimidazol-4-yl; and
R2 is hydroxy or, less preferably, hydrogen, and salts, especially pharmaceutically acceptable salts, thereof.

In a preferred embodiment of the invention, the Nanoparticles contain an entrapped drug for treatment of a solid tumor, such as zoledronic acid.

The outer surface of the Nanoparticle may contain a surface coating of hydrophilic polymers comprised of hydrophilic polymer chains, which are preferably densely packed to form a brushlike coating effective to shield Nanoparticle surface components. According to the invention, the hydrophilic polymer chains are connected to the Nanoparticle polymers chemically or adsorbed without any chemical linkage.

The outer surface of Nanoparticle may contain affinity moieties, effective to bind specifically to a target, e.g., a biological surface such as a cell membrane, a cell matrix, a tissue or target surface or region at which the Nanoparticle-based therapy is aimed. The affinity moiety is bound to the outer Nanoparticle surface by covalent attachment, as well as electrostatic interactions to the surface components and/or to the hydrophilic polymer coat in the Nanoparticles. The affinity moiety is a ligand effective to bind specifically and with high affinity to ligand-binding molecules carried on the target. For example, in one embodiment, the affinity moiety is effective to bind to a tumor-specific antigen and/or receptors over expressed in a solid tumor and in another embodiment, the affinity moiety is effective to bind to cells at a site of inflammation. In another embodiment, the affinity moiety is a vitamin, polypeptide or polysaccharide or protein effector.

The Nanoparticle of the present invention are for use in administering a therapeutic agent to a target. The therapeutic agent is entrapped within the Nanoparticle.

The Nanoparticle composition of the present invention is composed primarily of a polymer matrix. Such a polymer matrix one which:

(a) can be formed by emulsification;

(b) precipitation or surface deposition method; or

(c) can be formed by other nanoparticle manufacturing methods known in the art.

The Nanoparticle matrix forming polymers include polylactide, polyglycolide and copolymers of the aforementioned polymers (commonly known as poly lactic glycolic acids or PLGA), poly aminoacids, copolymers of polyaminoacids, glycosamino glycans, lipidated glycosaminoglycans etc.

Additionally, the polymer is selected to achieve a specified degree of fluidity or rigidity, to control the stability of the Nanoparticle in serum and to control the rate of release of the entrapped agent in the Nanoparticle. The rigidity of the Nanoparticle, as determined by the polymer, may also play a role in fusion of the Nanoparticle to a target cell, as will be described.

The Nanoparticles of the invention may contain a hydrophilic polymer coating made up of polymer chains which are linked to Nanoparticle surface. Such hydrophilic polymer chains are incorporated in the Nanoparticle by including between about 1-20 mole percent hydrophilic polymer-polymer matrix conjugate. Hydrophilic polymers suitable for use in the polymer coating include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyglycerine and polyaspartamide, hyaluronic acid, polyoxyethlene-polyoxypropylene copolymer (poloxamer), lecithin, polyvinyl alcohol.

In a preferred embodiment, the hydrophilic polymer is polyethyleneglycol (PEG), preferably as a PEG chain having a molecular weight between 500-10,000 daltons, more preferably between 2,000-10,000 daltons and most preferably between 1,000-5,000 daltons.

In another preferred embodiment, the hydrophilic polymer is polyglycerine (PG), preferably as a PG chain having a molecular weight between 400-2000 daltons, more preferably between 500-1,000 daltons and most preferably between 600-700 daltons.

The Nanoparticle composition of the present invention may contain an affinity moiety. The affinity moiety is generally effective to bind specifically to a target, that is, a biological surface such as a target cell surface or membrane, cell surface receptors, a cell matrix, a region of plaque or the like. The affinity moieties are bound to the Nanoparticle surface by direct attachment to the polymer component of the polymer matrix or by attachment to the hydrophilic polymer chain, as will be described.

In one embodiment, the affinity moiety is a ligand effective to bind specifically with a receptor at the target region, more specifically, a ligand for binding to a receptor on a target cell. Non-limiting examples of ligands suitable for this purpose are listed in Table 1.

TABLE 1
Ligand-Receptor Pairs and Associated Target Cell

| | | Epithelial Carcinomas, |
|---|---|---|
| Folate | Folate Receptor | Bone Marrow Stem Cells |
| Water soluble vitamins | Vitamin receptor | Various cells |
| Pyridoxyl phosphate | CD4 | CD4 + lymphocytes |
| Apolipoproteins | LDL | Liver hepatocytes, |
| | | Vascular endothelial cells |
| Insulin | Insulin receptor | |
| Transferrin | Transferring | Endothelial cells (brain) |
| | receptor | |
| Galactose | Asialoglycoprotein | Liver hepatocytes |
| Sialyl-Lewis* | E, P selectin | Activated endothelial cells |
| VEGF | Flk-1,2 | Tumor epithelial cells |
| Basic FGF | FGF receptor | Tumor epithelial cells |
| EGF | EGF receptor | Epithelial cells |
| VCAM-1 | A₄β₂-integrin | Vascular endothelial cells |
| ICAM-1 | α_{L}β₂-integrin | Vascular endothelial cells |
| PECAM-1/CD31 | αᵥβ₃-integrin | Vascular endothelial cells |
| Fibronectin | αᵥβ₃-integrin | Activated platelets |
| Osteopontin | αᵥβ₁ and | Smooth muscle cells in |
| | aᵥβ₅-integrin | atherosclerotic plaques |
| RGD sequences of | αᵥβ₃-integrin | Tumor endothelial cells, |
| matrix proteins | | vascular smooth muscle |
| | | cells |

The ligands listed in Table 1 may be used, in one embodiment of the invention, to target the Nanoparticles, to specific target cells. For example, a folate ligand attached to the polymer in the polymer matrix or to the distal end of a PEG chain can be incorporated into the Nanoparticles. A PEG chain, as used herein, is meant to specify a PEG chain having a length (molecular weight) selected such that the ligand, when incorporated into the Nanoparticle, is masked or shielded by the surface coating of hydrophilic polymer chains. A surface-bound folate ligand incorporated onto the Nanoparticle is effective to bind to folate receptors on epithelial cells for administration of an entrapped therapeutic agent to the target cell, e.g., administration of a neoplastic agent for treatment of epithelial carcinomas.

The affinity moiety is a short peptide that has cell-binding activity and is effective to compete with a ligand for a receptor site. Inhibition of the ligand-receptor cell-binding event results in arresting an infection process.

Polymer matrices containing the entrapped agent are prepared according to well-known methods, such as those described above, typically, emulsion, double emulsion and microencapsulation. The compound to be delivered is either included in the organic medium, in the case of a lipophilic compound, or is included in the aqueous medium, in the case of a water-soluble therapeutic agent. Alternatively, the therapeutic agent may be loaded into preformed matrices prior to administration to the subjects.

### II. Nanoparticle Preparation

### A. Preparation of Releasable Polymer Coating

The hydrophilic polymer chains are attached to the Nanoparticle through a linkage, that may cleave in response to a selected stimulus. In one embodiment, the linkage is a peptide, ester or disulfide linkage.

A peptide-linked compound is prepared, e.g., by coupling a polyalkylether, such as PEG, to a amine. End-capped PEG is activated with a carbonyl diimidazole coupling reagent, to form the activated imidazole compound. The activated PEG is then coupled to with the N-terminal amine of the exemplary tripeptide shown. The peptide amine group can then be used to couple a carboxyl group, through a conventional carbodiimide coupling reagent, such as dicyclohexylcarbodiimide (DCC).

The ester linked compound can be prepared, e.g., by coupling a polymer acid, such as polylactic acid, to the terminal alcohol group of a polyalkylether, using alcohol via an anhydride coupling agent. Alternatively, a short linkage fragment containing an internal ester bond and suitable end groups, such as primary amine groups, can be used to couple the polyalkylether to the matrix-forming polymer through amide or carbamate linkages.

### B. Attachment of Affinity Moiety

As described above, the Nanoparticles of the present invention may contain an affinity moiety attached to the surface of the PEG-coated Nanoparticles. The affinity moiety is attached to the Nanoparticles by direct attachment to Nanoparticle surface components or through a short spacer arm or tether, depending on the nature of the moiety.

A variety of methods are available for attaching molecules, e.g., affinity moieties, to the surface of polymer matrices. In one preferred method, the affinity moiety is coupled to the polymer, by a coupling reaction described below, to form an affinity moiety-polymer conjugate. This conjugate is used for formation of Nanoparticles. In another method, a matrix-forming polymer activated for covalent attachment, or other interaction (i.e., electrostatic) of an affinity moiety is incorporated into Nanoparticles.

In general, attachment of a moiety to a spacer arm can be accomplished by derivatizing the matrix-forming polymer, typically PLGA, with a hydrophilic polymer, such as PEG, having a reactive terminal group for attachment of an affinity moiety. Methods for attachment of ligands to activated PEG chains are described in the art (Allen, et al., 1995; Zalipsky, 1993; Zalipsky, 1994; Zalipsky, 1995a; Zalipsky, 1995b). In these methods, the inert terminal methoxy group of mPEG is replaced with a reactive functionality suitable for conjugation reactions, such as an amino or hydrazide group. The end functionalized PEG is attached to a lipid, typically DSPE. The functionalized PEG-polymer derivatives are employed in Nanoparticle formation and the desired ligand is attached to the reactive end of the PEG chain before or after Nanoparticle formation. In the aforementioned approach, the efficiency of covalent linkage to the polymer component has to be established depending the polymer used. Therefore, in another approach, a bifunctional polymer can be used to covalently link a targeting moiety on one end and a charge moiety on the other end. The charge moiety is selected such that its charge is opposite to that of the polymer component used for forming the polymer matrix.

### C. Nanoparticle Preparation

The Nanoparticles may be prepared by a variety of techniques, such as emulsion or double emulsion. Typically, the polymer is dissolved in an organic solvent and the drug is dissolved either in the organic solvent or the aqueous phase depending on its relative solubility in these two phases. An oil in water emulsion is formed and the solvent diffuses out rapidly allowing the polymer to precipitate as nanoparticles. This process is generally applicable to hydrophobic drugs that are soluble in the same solvent as the polymer. For hydrophilic drugs, a water in oil in water double emulsion (w/o/w) process can be employed. The particle size is determined by the energy input such as by sonication.

The matrix polymers used in forming the Nanoparticles of the present invention are preferably present at about 20-98% of the matrix.

Still another Nanoparticle preparation procedure suitable for preparation of the Nanoparticles of the present invention is a solvent injection method. In this procedure, a mixture of the polymers, dissolved in a solvent, is injected into an aqueous medium with stirring to form Nanoparticles. The solvent is removed by a suitable technique, such as dialysis or evaporation.

The Nanoparticles are preferably prepared to have substantially homogeneous sizes in a selected size range, typically between about 10 nm to about 500 nm, preferably 50 nm to about 300 nm and most preferably 80 nm to about 200 nm.

When desired, the Nanoparticles can be dried, such as by evaporation or lyophilization and resuspended in any desirable solvent. Where Nanoparticles are lyophilized, non-reducing sugars can be added prior to lyophilization or during Nanoparticle formulation to provide stability. One such sugars are mannitol, sucrose, trehlaose. Other stabilizing agents can include amino acids, i.e., glycine.

The Nanoparticle having a divalent cation matrix can be made by an addition of a solvent containing a divalent cation during Nanoparticle preparation.

The Nanoparticles can be resuspended into the aqueous solution by gentle swirling of the solution. The rehydration can be performed at room temperature or at other temperatures appropriate to the composition of the Nanoparticles and their internal contents.

### III. Method of Treatment

The invention includes, in one aspect, a method of Nanoparticle-based therapy for a mammalian subject which includes systemically administering to the subject, Nanoparticles containing:
(i) a divalent cation matrix; and
(ii) a therapeutic agent.
   The divalent cation matrix provides protection of a therapeutic agent which otherwise might leak out of traditional liposomal formulation on the shelf and once introduced into the body. Another aspect, the invention includes a method of Nanoparticle-based therapy for a mammalian subject which includes systemically administering to the subject Nanoparticles containing:
(i) a divalent cation matrix;
(ii) a therapeutic agent;
(iii) a hydrophilic polymer coating for stability and prolonged circulation; and, optionally
(iv) an affinity moiety effective to bind specifically to a target surface at which the therapy is aimed.
   The hydrophilic polymer coating is made up of polymer chains which are either covalently linked or surface adsorbed to surface polymer components in the Nanoparticles. The administered Nanoparticles are allowed to circulate systemically until a desired biodistribution of the Nanoparticles is achieved, thereby to expose the affinity agent to the target surface.

In a preferred embodiment, the Nanoparticles are used for treatment of a solid tumor. The Nanoparticles include an anti-tumor drug in entrapped form and are targeted to the tumor region by an affinity moiety effective to bind specifically to a tumor-specific antigen. For example, Nanoparticles can be targeted to the vascular endothelial cells of tumors by including a VEGF ligand in the Nanoparticle, for selective attachment to Flk-1,2 receptors expressed on the proliferating tumor endothelial cells.

In this embodiment, the Nanoparticles are sized to between about 10-200 nm, preferably 50-150 nm and most preferably 80-120 nm. Nanoparticles in this size range have been shown to be able to enter tumors through “gaps” present in the endothelial cell lining of tumor vasculature [Yuan, et al. (1995)].

In one embodiment, the therapeutic agents are selected from the compounds of formula (I). The compounds of formula (I), and salts thereof, have valuable pharmacological properties. In particular, they have a pronounced regulatory action on the calcium metabolism of warm-blooded animals. Most particularly, they effect a marked inhibition of bone resorption in rats, as can be demonstrated in the experimental procedure described in Acta Endrocinol, Vol. 78, pp. 613-24 (1975), by means of the PTH-induced increase in the serum calcium level after subcutaneous administration of doses in the range from about 0.01-1.0 mg/kg, as well as in the TPTX (thyroparathyroidectomised) rat model by means of hypercalcaemia induced by vitamin D₃ after subcutaneous administration of a dose of about 0.0003-1.0 mg. Tumor calcaemia induced by Walker 256 tumors is likewise inhibited after peroral administration of about 1.0-100 ma/kg. In addition, when administered subcutaneously in a dosage of about 0.001-1.0 mg/kg in the experimental procedure according to Newbould, Brit J Pharmacol, Vol. 21, p. 127 (1963), and according to Kaibara et al., J Exp Med, Vol. 159, pp. 1388-96 (1984), the compounds of formula (I), and salts thereof, effect a marked inhibition of the progression of arthritic conditions in rats with adjuvant arthritis. They are therefore eminently suitable for use as medicaments for the treatment of diseases which are associated with impairment of calcium metabolism, e.g., inflammatory conditions in joints, degenerative processes in articular cartilege, of osteoporosis, periodontitis, hyperparathyroidism, and of calcium deposits in blood vessels or prothetic implants. Favorable results are also achieved in the treatment of diseases in which an abnormal deposit of poorly soluble calcium salts is observed, as in arthritic diseases, e.g., ancylosing spondilitis, neuritis, bursitis, periodontitis and tendinitis, fibrodysplasia, osteoarthrosis or arteriosclerosis, as well as those in which an abnormal decomposition of hard body tissue is the principal symptom, e.g., heriditary hypophosphatasia, degenerative states of articular cartilege, osteoporosis of different provenance, Paget's disease and osteodystrophia fibrosa, and also osteolytic conditions induced by tumors.

After administration of the Nanoparticles, e.g., intravenous administration, and after sufficient time has elapsed to allow the Nanoparticles to distribute through the subject and extravasate into the tumor, the affinity moiety of the Nanoparticles provides binding and internalization into the target cells. In one embodiment, the hydrophilic surface coating is attached to the Nanoparticles by a pH sensitive linkage, and the linkages are released after the Nanoparticles have extravasated into the tumor, due to the hypoxic nature of the tumor region.

From the foregoing, it can be appreciated how various features and objects of the invention are met. The Nanoparticles of the present invention provide a method for targeting Nanoparticles. The hydrophilic surface coating reduces uptake of the Nanoparticles, achieving a long blood circulation lifetime for distribution of the Nanoparticles. After distribution, the Nanoparticle-attached affinity moieties allow for multi-valent presentation and binding with the target.

The following examples illustrate methods of preparing, characterizing, and using the Nanoparticles of the present invention. The examples are in no way intended to limit the scope of the invention. Although the invention has been described with respect to particular embodiments, it will be apparent to those skilled in the art that various changes and modifications can be made without departing from the invention.

### Examples

In the following examples, Nanoparticles were prepared by the double emulsion method. All samples were processed by sonication, evaporation, centrifugation and lyophilization in the presence of water or 5% mannitol (or other suitable bulking agent, i.e., sucrose).

The examples below were manufactured without any divalent cation and this formulation provided very low drug loading in the matrix.

### Example A

| | |
|---|---|
| 1. ZOL446 | 30 mg/mL (2.8% PVA/tris buffer pH 8) |
| 2. PLGA, 50:50, 90,000 MW | 30 mg/mL (in methylene chloride) |
| 3. PVA | 3% (tris buffer pH 8 + calcium chloride) |

The drug solution in step 1 is added to the polymer solution in step 2 by sonication. This primary emulsion is added to the PVA solution in step 3 and the sonication is continued. The nanoparticles are harvested by evaporation of the solvent, washing and centrifugation. The product is lyophilized in the presence of water or 5% mannitol.

### Example B

| | |
|---|---|
| 1. ZOL446 | 30 mg/mL (2.8% PVA/tris buffer pH 8) |
| 2. PLGA, 50:50, 50,000 MW | 30 mg/mL (in methylene chloride) |
| 3. PVA | 3% (tris buffer pH 8 + calcium chloride) |

The drug solution in step 1 is added to the polymer solution in step 2 by sonication. This primary emulsion is added to the PVA solution in step 3 and the sonication is continued. The nanoparticles are harvested by evaporation of the solvent, washing and centrifugation. The product is lyophilized in the presence of water or 5% mannitol.

### Example C

| | |
|---|---|
| 1. ZOL446 | 30 mg/mL (2.8% PVA/tris buffer pH 8) |
| 2. PLGA, 50:50, 10,000 MW | 30 mg/mL (in methylene chloride) |
| 3. PVA | 2% (tris buffer pH 8 + calcium chloride) |

The drug solution in step 1 is added to the polymer solution in step 2 by sonication. This primary emulsion is added to the PVA solution in step 3 and the sonication is continued. The nanoparticles are harvested by evaporation of the solvent, washing and centrifugation. The product is lyophilized in the presence of water or 5% mannitol.

### Example D

| | |
|---|---|
| 1. ZOL446 | 10 mg/mL (1% PVA/tris buffer pH 8) |
| 2. PLGA, 50:50, 10,000 MW | 50 mg/mL (in ethyl acetate) |
| 3. PVA | 5% (tris buffer pH 8 + calcium chloride) |

The drug solution in step 1 is added to the polymer solution in step 2 by sonication. This primary emulsion is added to the PVA solution in step 3 and the sonication is continued. The nanoparticles are harvested by evaporation of the solvent, washing and centrifugation. The product is lyophilized in the presence of water or 5% mannitol.

### Example E

| | |
|---|---|
| 1. ZOL446 | 30 mg/mL (2.8% PVA/tris buffer pH 8) |
| 2. PLGA, 50:50, 90,000 MW | 30 mg/mL (in methylene chloride) |
| 3. PVA | 3% (tris buffer pH 8) |

The drug solution in step 1 is added to the polymer solution in step 2 by sonication. This primary emulsion is added to the PVA solution in step 3 and the sonication is continued. The nanoparticles are harvested by evaporation of the solvent, washing and centrifugation. The product is lyophilized in the presence of water or 5% mannitol.

### Example F

| | |
|---|---|
| 1. ZOL446 | 10 mg/mL (2.8% PVA/tris buffer pH 8) |
| 2. PLGA, 50:50, 140,000 MW | 50 mg/mL (in ethyl acetate) |
| 3. PVA | 1% (tris buffer pH 8 + |
| | calcium chloride) |

The drug solution in step 1 is added to the polymer solution in step 2 by sonication. This primary emulsion is added to the PVA solution in step 3 and the sonication is continued. The nanoparticles are harvested by evaporation of the solvent, washing and centrifugation. The product is lyophilized in the presence of water or 5% mannitol.

### Example G

| 1. ZOL446 | 0.4 mg/mL (1% poloxamer, 0.1 N HCl) |
|---|---|
| 2. PLGA, 50:50, 75,000 MW | 4 mg/mL (in acetone) |
| 3. Poloxamer | 1% |

The polymer solution in step 2 is added to the drug solution in step 1 by mixing. Evaporate the acetone and collect the nanoparticles. The product is lyophilized in the presence of 5% mannitol.

It is claimed:

1. A method of administering zoledronic acid to a mammalian subject, comprising systemically administering a nanoparticle composition comprising a PLGA polymer matrix with calcium ions wherein the PLGA polymer matrix contains zoledronic acid.

2. The method of claim 1, wherein the nanoparticle composition has an average particle size of about 10 nanometers (nm) to about 500 nm.

3. The method of claim 1, wherein the nanoparticle composition further comprises a hydrophilic polymer.

4. The method of claim 1, wherein the nanoparticle composition further comprises an affinity moiety.

5. A nanoparticle composition comprising a PLGA polymer matrix with calcium ions wherein the PLGA polymer matrix contains zoledronic acid as a therapeutic agent.

6. The nanoparticle composition of claim 5 further comprising a hydrophilic polymer.

7. The nanoparticle composition of claim 5 further comprising an affinity moiety.

8. A nanoparticle composition comprising:
(a) zoledronic acid as a therapeutic agent;
(b) a PLGA polymer matrix with calcium ions;
(c) a hydrophilic polymer coating; and
(d) an affinity moiety.

9. The nanoparticle composition of claim 8, wherein the affinity moiety is a ligand effective to bind specifically with a cell-surface receptor on the target surface.

10. The nanoparticle composition of claim 8, wherein the affinity moiety is effective to bind specifically to a tumor-specific receptor and/or antigen.

11. The nanoparticle composition of claim 8 having an average particle size of about 10 nm to about 500 nm.
